(19) United States
(12) Patent Application Publication
(10) Pub. No.: US 2009/0209005 A1
(43) Pub. Date: Aug. 20, 2009

(54) Chimeric dna polymerase

(76) Inventors: Konstantin Ignatov, Moscow (RU); Vladimir Kramarov, Moscow (RU); Sam Billingham, Greater London (GB)

Correspondence Address: NIXON & VANDERHYE, PC, 901 NORTH GLEBE ROAD, 11TH FLOOR, ARLINGTON, VA, 22203, US

(21) Appl. No.: 11/658,610

(22) PCT Filed: Jul. 14, 2005
(86) PCT No.: PCT/GB05/02786

§ 371 Date: Jan. 26, 2007

(30) Foreign Application Priority Data
Jul. 26, 2004 (GB) 0416610.4

(51) Int. Cl. C12P 21/00; C12N 9/10; C07H 21/04; C12N 15/74; C12N 5/06; C12P 19/34

(52) U.S. Cl. 435/69.7; 435/193; 536/23.2; 435/320.1; 435/325; 435/91.2

(57) ABSTRACT

The present invention provides a chimeric thermostable DNA polymerase that includes a region from a Tth DNA polymerase I, a region from a Taq DNA polymerase I and a DNA polymerase domain. The DNA polymerase domain comprises a portion of a DNA polymerase domain from the Tth DNA polymerase I operably linked to a portion of a DNA polymerase domain from the Taq DNA polymerase I. Also provided are a nucleic acid sequence and an amino acid sequence of the chimeric thermostable enzyme of the invention. The chimeric DNA polymerase enzyme of the invention is useful in DNA amplification reactions such as the polymerase chain reaction.

### FIELD OF THE INVENTION

The present invention relates to thermostable DNA polymerases, polynucleotide and amino acid sequences encoding them, their synthesis and methods for their use.

### BACKGROUND ART

Thermostable DNA polymerases are well known, and are useful in a wide range of laboratory processes, especially in molecular biology. Primer extension techniques, nucleic acid sequencing and the polymerase chain reaction (PCR) all employ such enzymes.

DNA polymerases, which catalyze the template-directed polymerization of deoxyribonucleoside triphosphates (dNTPs) to form DNA, are used in a variety of in vitro DNA synthesis applications, such as primer extension techniques, DNA sequencing and DNA amplification.

Thermostable DNA polymerases are particularly useful in a number of these techniques, as thermostable enzymes can be used at relatively high temperatures. This has benefits with respect to fidelity of primer binding, for example, owing to the high stringency of the conditions employed. Of known enzymes, the DNA polymerases isolated from Thermus aquaticus (Taq) and Thermus thermophilus (Tth) are perhaps the best characterized.

These enzymes have a defined range of different properties and limitations. For example, Taq and Tth DNA polymerases differ from each other in the following practically significant properties:

1) Tth DNA polymerase is more effective than Taq DNA polymerase for amplification of long (over 2 kb) DNA sequences in PCR [Ohler L. D., and Rose E. A., PCR Methods Appl. V.2 (1992), P. 51-59; Ignatov K. B. et al., Mol. Biol. (Russ.) V.31 (1997), P. 956-961] which is seen as a larger quantity of DNA produced;

2) Taq DNA polymerase is more sensitive than Tth DNA polymerase to the presence of a mismatched (non-complementary to template) nucleotide at the 3′-end of the primer [Ignatov K. B. et al., Bioorg. Khim. (Russ.) V.23 (1997), P. 817-822], which allows to employ Taq DNA polymerase in allele-specific primer extension reactions;

3) Taq DNA polymerase is more specific than Tth DNA polymerase in DNA amplification in the course of PCR [Ignatov K. B. et al., Bioorg. Khim. (Russ.) V.23 (1997), P. 817-822], and thus yields a higher ratio of target product to total synthesized DNA.

For increasing the efficiency of laboratory processes employing the above-mentioned DNA polymerases, a creation of a novel DNA polymerase that would possess the advantages and lack the drawbacks of these enzymes is deemed very useful. For instance, primer extension techniques (such as allele-specific primer extension) and PCR-amplification of DNA would need a thermostable DNA polymerase combining the efficiency of DNA synthesis of Tth DNA polymerase and the specificity of PCR-based DNA amplification characteristic of Taq DNA polymerase.

It has been shown earlier that combining in one polypeptide chain portions of protein molecules from different DNA polymerases may lead to construction of chimeric DNA polymerases having a combination of properties possessed by the parental DNA polymerases [Ignatov K. B. et al., Mol. Biol. (Russ.) V.31 (1997), P. 956-961; Villbrandt B. et al., Protein Eng. V.13 (2000), P. 645-654; U.S. Pat. No. 6,228,628; U.K. Patent No. GB2344591].

The N-terminal region of Taq DNA polymerase has been shown to exert a significant effect on the efficiency of PCR with DNA templates longer than 2 kb. For example, deletion of the first 235 amino acids of Taq DNA polymerase reduces the enzyme's ability to amplify long DNA sequences [Barnes W. M., Gene V.112 (1992), P. 29-35]. The ability of Taq and Tth DNA polymerases to amplify long DNA sequences has also been attributed to sequences between the corresponding amino acid positions 498 and 554 for Taq DNA polymerase and 500 and 556 for Tth DNA polymerase [Blanco L. et al., Gene V.100 (1991), P. 27-28; Ignatov K. B. et al., Mol. Biol. (Russ.) V.31 (1997), P. 956-961]. Differences in those regions have, however, no effect on the specificity of DNA synthesis and the sensitivity of the two DNA polymerases to the presence of a mismatch at the 3′-end of the primer [Ignatov K. B. et al., Mol. Biol. (Russ.) V.31 (1997), P. 956-961].

The above-mentioned earlier findings have allowed us to make the conclusion that combining in one polypeptide chain the N-terminal region of Tth DNA polymerase (including the region spanning amino acids 500-556) with the C-terminal region of Taq polymerase (containing the sequence corresponding to amino acids 600-832 of the Taq sequence) will allow us to obtain a chimeric thermostable DNA polymerase possessing the synthesis efficiency of Tth DNA polymerase and the specificity of Taq DNA polymerase. Thus, this chimeric DNA polymerase with a combination of desirable properties that do not occur in nature would be useful in a variety of in vitro DNA synthesis applications.

### SUMMARY OF THE INVENTION

The present invention relates to a chimeric thermostable enzyme, which has DNA polymerase activity.

In a first embodiment the present invention provides a chimeric thermostable enzyme comprising an N-terminal region, a C-terminal region and a DNA polymerase domain. The N-terminal region of the chimeric thermostable enzyme comprises an N-terminal region from a Tth DNA polymerase I and the C-terminal region of the chimeric thermostable enzyme comprises a C-terminal region from a Taq DNA polymerase I. The DNA polymerase domain comprises a portion of a DNA polymerase domain of the N-terminal region from a Tth DNA polymerase I operably linked to a portion of a DNA polymerase domain of the C-terminal region from a Taq DNA polymerase I.

The chimeric thermostable enzyme according to the present invention may also include a portion of, or all of, a 5′ nuclease domain located in the N-terminal region from the Tth DNA polymerase I. The inclusion of a 5′ nuclease domain confers 5′ to 3′ nuclease activity on the chimeric thermostable enzyme.

Preferably the chimeric thermostable enzyme of the present invention comprises the amino acid sequence of SEQ ID No: 1, or a variant or fragment thereof as defined herein below.

Preferably a variant of SEQ ID NO: 1 has at least 92%, sequence identity to SEQ ID No: 1, for example 93%, 94%, 95%, 96%, 97%, 98% or 99% sequence identity. Thus for example the variant may differ from the sequence set out as SEQ ID NO: 1 by one or more of addition, substitution, deletion and insertion, preferably conservative substitution, of one or more (such as from 1 or 2, 3, 4, 5, 6, 7, 8, or 9, or about 10, 12, 14, 16, 18 or 19) amino acids. The variant will retain DNA polymerase I activity and may retain 5′ nuclease activity.

The invention also provides a fragment of SEQ ID NO: 1, or a fragment of the above-mentioned variant of SEQ ID NO: 1. Such a fragment may comprise residues 280 to 828 of SEQ ID NO: 1 or its variant when aligned with SEQ ID NO: 1. The fragment retains DNA polymerase I activity and may retain 5′ nuclease activity.

For example, in the chimeric thermostable enzyme of SEQ ID NO: 1, the Asp found at position 2 of the Tth DNA polymerase I sequence of SEQ ID NO: 13 has been be substituted with Glu, and the Leu at position 3 of the Tth DNA polymerase I of SEQ ID NO: 13 sequence has been substituted with Ala in the chimeric thermostable enzyme of the present invention. Thus residues 2 and 3 of SEQ ID NO: 1 may be varied to be Asp and Leu respectively. Other variants are discussed further herein below.

In one embodiment the invention provides a chimeric thermostable enzyme comprising an N-terminal region, a C-terminal region and a DNA polymerase domain, wherein the N-terminal region comprises an N-terminal region from a Thermus thermophilus (Tth) DNA polymerase I, the C-terminal region comprises a C-terminal region from a Thermus aquaticus (Taq) DNA polymerase I and the DNA polymerase domain comprises a portion of a DNA polymerase domain of the N-terminal region from Tth DNA polymerase I operably linked to a portion of a DNA polymerase domain of the C-terminal region from Taq DNA polymerase I, wherein the N-terminal region of the chimeric enzyme comprises an amino acid sequence from between positions 1 to 280 through to position n of Tth DNA polymerase I (SEQ ID No: 13), wherein n is between amino acids 555 to 601 of Tth DNA polymerase I and corresponds to an amino acid in position m of Taq DNA polymerase I (SEQ ID No: 14), wherein m is equal to n−2.

The present invention provides a nucleic acid encoding a chimeric thermostable enzyme of the invention. Preferably the nucleic acid has the nucleotide sequence as shown in SEQ ID No: 2.

A further embodiment of the present invention relates to a recombinant DNA vector that contains the nucleic acid sequence encoding the chimeric thermostable enzyme of the invention operably linked to a promoter, and a host cell transformed with the recombinant DNA vector. Also encompassed is a method of making a chimeric thermostable enzyme of the invention comprising cultivating the host cell of the invention under conditions for expression of said enzyme, and recovering said enzyme from the cell.

In another embodiment of the present invention, a kit is provided which may comprise a chimeric thermostable enzyme of the invention as well as a reaction buffer and dNTPs. Such a kit, along with primers and double stranded template DNA, may be used in the technique of DNA amplification using the polymerase chain reaction (PCR).

### BRIEF DESCRIPTION OF THE FIGURES AND TABLES

FIG. 1 provides a scheme illustrating steps in construction of chimeric gene encoding the chimeric polymerase of the invention and an expression vector.

FIG. 2 provides a photograph of an agarose gel, which compares the yield of 2500-bp DNA fragment obtainable by PCR amplification with Taq DNA polymerase, Tth DNA polymerase and the chimeric DNA polymerase of this invention.

FIG. 3 provides a photograph of an agarose gel, which compares the specificity of PCR amplification reactions performed with Taq DNA polymerase, Tth DNA polymerase and the chimeric DNA polymerase of this invention.

TABLE 1 provides data of radioactive label incorporation into the 500-bp DNA fragment synthesized with Taq, or Tth, or the chimeric DNA polymerase by PCR with primers containing or not containing 3′-mismatching nucleotides

### DETAILED DESCRIPTION OF THE INVENTION

The present invention provides a chimeric thermostable DNA polymerase and means for producing the enzyme. To facilitate understanding of the invention, a number of terms are defined below.

Chimeric

The term “chimeric” in the context of the present invention is used with reference to an enzyme whose amino acid sequence comprises subsequences of amino acid sequences from at least two distinct proteins. These subsequences can be operably linked to produce the chimeric enzyme. By operably linked is meant the joining of constituent subsequences such that a functional enzyme is obtained. The linkage may be achieved by a variety of methods such as ligation.

Thermostable Enzyme

Thermostable enzymes are well known in the art. The term “thermostable enzyme”, as used herein, refers to an enzyme which is stable to heat and reacts optimally at an elevated temperature. The thermostable enzyme of the present invention catalyses primer extension optimally at a temperature between 60 and 90° C., and is usable under the temperature cycling conditions typically used in cycle sequence reactions and polymerase chain reaction amplifications (described in U.S. Pat. No. 4,965,188).

N-Terminal Region from a Tth DNA Polymerase I

By “N-terminal region from a Tth DNA polymerase I” it is meant the amino acid sequence (a) corresponding to position 1 to 601 of SEQ ID No: 13; (b) a variant which has at least 87%, 90%, 95%, 96%, 97%, 98% or 99% identity with (a); or (c) a C-terminal fragment of (a) or (b) with an N-terminus starting at a position corresponding to a residue from 4 to 280 of SEQ ID NO: 13. The variant (b) may have a sequence which, for example, corresponds to (a) apart from a change of a single amino acid or 2, 3, 4, 5, 6, 7, 8, or 9 changes, or about 10, 15, 20, 30, 40, 50, 60 or 70 changes. Preferably the variant will have from 1 to 10 amino acid changes, for example from 1 to 5 changes. The variant may be produced by means of addition, substitution, deletion and insertion of one or more amino acids, preferably by means of a conservative substitution, as defined below. The fragment (c) may comprise, as a minimum sequence, an amino acid sequence from positions 280 to 555 of SEQ ID No: 13. The variants and fragments when linked to the C-terminal region of a Taq DNA polymerase I will exhibit DNA polymerase I activity. The variants and fragments may also exhibit 5′-nuclease activity.

In a preferred embodiment, the N-terminal region from a Tth DNA polymerase I comprises an amino acid sequence from between positions 1 to 280 through to a position n of a Tth DNA polymerase I and includes a portion of a DNA polymerase domain. Position n may be between amino acids 555 to 601 of a Tth DNA polymerase I and corresponds to an amino acid in position m of a Taq DNA polymerase I, wherein m is equal to n-2. Preferably, the N-terminal region from a Tth DNA polymerase I comprises an amino acid sequence from between positions 4 to 600 of SEQ ID No: 13.

C-Terminal Region from a Taq DNA Polymerase I

By “C-terminal region from a Taq DNA polymerase I” it is meant the amino acid sequence (a) corresponding to position m+1 to 832 of SEQ ID No: 14; (b) a variant which has at least 92%, 93%, 94%, 95%, 96%, 97%, 98% or 99% identity with (a); or (c) a fragment of (a) or (b) having an amino acid sequence corresponding to position m+1 to between 826 to 832 of SEQ ID NO: 14. Position m may be between amino acids 553 to 598 of a Taq DNA polymerase I and corresponds to an amino acid in position n of a Tth DNA polymerase I, wherein n is equal to m+2. The variant (b) may have a sequence which, for example, corresponds to (a) apart from a change of a single amino acid or 2, 3, 4, 5, 6, 7, 8, or 9 changes, or about 10, 12, 14, 16, 18 or 19 changes. Preferably the variant will have from 1 to 10 amino acid changes, for example from 1 to 5 changes. The variant may be produced by means of addition, substitution, deletion and insertion of one or more amino acids, preferably by means of a conservative substitution, as defined below. The fragment (c) may comprise, as a minimum sequence, an amino acid sequence from positions 599 to 826 of SEQ ID No: 14. The variants and fragments, when linked to the N-terminal region of a Tth DNA polymerase I, will exhibit DNA polymerase I activity.

In a preferred embodiment, the C-terminal region from a Taq DNA polymerase I comprises an amino acid sequence from a position m+1 through to 832 of a Taq DNA polymerase I and includes a portion of a DNA polymerase domain. Preferably, the C-terminal region from a Taq DNA polymerase I comprises an amino acid sequence from between positions 554 to 832 of SEQ ID No: 14.

Structure of a Chimeric Thermostable Enzyme

Preferably, the chimeric thermostable enzyme of the invention comprises an amino acid sequence (a) corresponding to SEQ ID No: 1; (b) a variant which has at least 92%, 93%, 94%, 95%, 96%, 97%, 98% or 99% identity with (a); or (c) a fragment of (a) or (b). The variant (b) may have a sequence which, for example, corresponds to (a) apart from a change of a single amino acid or 2, 3, 4, 5, 6, 7, 8, or 9 changes, or about 10, 12, 14, 16, 18 or 19 changes. Preferably the variant will have from 1 to 10 amino acid changes, for example from 1 to 5 changes. The variant may be produced by means of addition, substitution, deletion and insertion of one or more amino acids, preferably by means of a conservative substitution, as defined below. The variants and fragments will exhibit DNA polymerase I activity and may exhibit 5′-nuclease activity.

Fragments of the invention may comprise about 550, 600, 650, 700, 750 or 800 amino acids. Preferably, fragments of a chimeric thermostable enzyme comprise an N-terminal region, a C-terminal region and a DNA polymerase domain. For example, an N-terminal region from a Tth DNA polymerase I or a portion thereof and a C-terminal region of a Taq DNA polymerase I or a portion thereof, as described above.

More preferably, the chimeric thermostable enzyme of the present invention comprises the sequence shown in SEQ ID No: 1 or a variant or fragment thereof.

Conservative Substitutions

Examples of conservative substitutions referred to above include those set out in the following table, where amino acids on the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |
| OTHER | | N Q D E |

Amino Acid Identity

The percentage identity of amino acid sequences can be calculated using commercially available algorithms. The following programs (provided by the National Center for Biotechnology Information) may be used to determine homologies: BLAST, gapped BLAST, BLASTN and PSI-BLAST, which may be used with default parameters.

Portion of a DNA Polymerase Domain

A “portion of a DNA polymerase domain” refers to a sequence of amino acids which form part of a DNA polymerase domain from a Tth DNA polymerase I and/or a Taq DNA polymerase I. In the present invention preferably, a portion of a DNA polymerase domain of the N-terminal region from a Tth DNA polymerase I is operably linked to a portion of a DNA polymerase domain of the C-terminal region from a Taq DNA polymerase I.

Cell

As used herein, “cell”, “cell line”, and “cell culture” can be used interchangeably and all such designations include progeny. Thus, the words “transformants” or “transformed cells” includes the primary subject cell and cultures derived therefrom without regard for the number of transfers. It is also understood that all progeny may not be precisely identical in DNA content, due to deliberate or inadvertent mutations. Mutant progeny that have the same functionality as screened for in the originally transformed cell are included.

Gene

The term “gene” refers to a DNA sequence that comprises control and coding sequences necessary for the production of a recoverable bioactive polypeptide or precursor.

Oligonucleotides

The term “oligonucleotides” as used herein is defined as a molecule comprised of two or more deoxyribonucleotides or ribonucleotides. The exact size will depend on many factors, which in turn depends on the ultimate function or use of the oligonucleotide. Oligonucleotides can be prepared by any suitable method, including, for example, cloning and restriction of appropriate sequences and direct chemical synthesis by a method such as the phosphotriester method, the diethylphosphoramidite method, and the solid support method. A review of synthesis methods is provided in [Goodchild J., Bioconjug. Chem. V.1 (1990), P. 165-187].

Primer

The term “primer” as used herein refers to an oligonucleotide, which is capable of acting as a point of initiation of synthesis when placed under conditions in which primer extension is initiated. Synthesis of a primer extension product, which is complementary to a nucleic acid strand, is initiated in the presence of the requisite four different nucleoside triphosphates and a thermostable DNA polymerase in an appropriate buffer at a suitable temperature. A “buffer” includes cofactors (such as divalent metal ions) and salt (to provide the appropriate ionic strength), adjusted to the desired pH.

A primer that hybridizes to the non-coding strand of a gene sequence (equivalently, is a subsequence of the coding strand) is referred to herein as an “upstream” primer. A primer that hybridizes to the coding strand of a gene sequence is referred to herein as a “downstream” primer.

Restriction Endonucleases

The terms “restriction endonucleases” and “restriction enzymes” refer to enzymes, typically bacterial in origin, which cut double-stranded DNA at or near a specific nucleotide sequence.

Construction of a Chimeric Thermostable Enzyme

The present invention provides a chimeric thermostable enzyme which has the properties of high efficiency of long (over 2 kb) DNA sequences amplification in PCR, high sensitivity to the presence of a mismatched (non-complementary to template) nucleotide at the 3′-end of the primer, and high specificity in DNA amplification in the course of PCR. Said properties being derived from at least two different sources, wherein the properties are preferably in combination.

It will be appreciated that a chimeric protein may be constructed in a number of ways. The chimeric thermostable enzyme of the present invention may be produced by direct manipulation of amino acid sequences. In an alternative and preferred embodiment, the chimeric thermostable enzyme is expressed from a chimeric gene that encodes the chimeric amino acid sequence i.e. via the construction of a recombinant DNA molecule, followed by expression of the protein product.

Manipulation at the DNA level allows DNA fragments from different genes to be joined together by ligation, to form DNA encoding a chimeric polymerase. DNA fragments from different DNA polymerase genes may be obtained by DNA purification, followed by restriction enzyme digestion, PCR, or even direct DNA synthesis, for example. The protein may then be expressed from the DNA, using expression vectors maintained within host cells. DNA cloning, manipulation and protein expression are all standard techniques in the art, and details of suitable techniques may be found in Sambrook et al, Molecular cloning—A Laboratory Manual, 1989.

The present invention, therefore, also provides DNA encoding a chimeric thermostable enzyme, along with a vector containing this DNA, host cells containing this vector, and cultures of such cells, as well as methods for making the enzyme.

Methods of making the enzyme are well known in the art and include cultivating a host cell of the invention under conditions for expression of a chimeric thermostable enzyme, and recovering the enzyme from the host cell. “Recovering the enzyme” means the process of isolation and/or purification of the protein of the chimeric thermostable enzyme from the host cell. For example, purification can be achieved on the basis of the size, solubility, charge and/or specific binding affinity (e.g. by the use of an antibody) of the protein.

Generally, nucleic acid according to the present invention is provided as an isolate, in isolated and/or purified form, or free or substantially free of material with which it is naturally associated, except possibly one or more regulatory sequence(s) for expression. Nucleic acid may be wholly or partially synthetic and may include genomic DNA, cDNA or RNA.

DNA and vectors encoding all or part of an enzyme of the invention may suitably incorporate such control elements, such as start/stop codons, promoters etc, as are deemed necessary or useful, as the skilled person desires. Suitable constructs are illustrated in the accompanying Examples.

The chimeric gene is produced from the Tth DNA polymerase gene and the Taq DNA polymerase gene using standard gene manipulation techniques well known in the field of molecular biology, as described in Example 1.

The gene encoding Tth DNA polymerase, the nucleotide sequence of the Tth DNA polymerase gene, as well as the full amino acid sequence of the encoded protein, is described in U.S. Pat. No. 5,618,711.

The gene encoding Taq DNA polymerase, the nucleotide sequence of the Taq DNA polymerase gene, as well as the full amino acid sequence of the encoded protein, are described in [Lawyer, F. C. et al., J. Biol. Chem., 261, 11, 6427-6437] and U.S. Pat. No. 5,079,352.

Sequence of a Chimeric Thermostable Enzyme of Example 1

The amino acid sequence of a chimeric thermostable enzyme of the invention is given in SEQ ID No: 1. A part of the amino acid sequence of the chimeric thermostable enzyme from amino acids 4 through 600 comprises the sequence of amino acids 4-600 of Tth DNA polymerase I. A part of the amino acid sequence of the chimeric thermostable enzyme from amino acids 556 through 834 comprises the sequence of amino acids 554-832 of Taq DNA polymerase I. Thus, the sequence of amino acids 556-600 of the chimeric thermostable enzyme is identical to both the sequence of amino acids 556-600 of Tth DNA polymerase I and the sequence of amino acids 554-598 of Taq DNA polymerase I.

The sequence of amino acids 1-3 of the chimeric thermostable enzyme of the invention arose from recombinant expression vector construction (described in Example 1).

Nucleic Acid Encoding the Chimeric Thermostable Enzyme of Example 1

The nucleotide sequence of the nucleic acid encoding a chimeric thermostable enzyme is given in SEQ ID No: 2. The nucleic acid encoding the chimeric thermostable enzyme was obtained as described in Example 1.

The nucleotide sequence of the nucleic acid encoding the chimeric DNA polymerase consists of subsequences:
the sequence of nucleotides 1-8, which arose from recombinant expression vector construction (described in Example 1);
the sequence of nucleotides 9-1786, which was taken from the gene of Tth DNA polymerase, and which is identical to the nucleotide sequence 9-1786 of Tth DNA polymerase gene;
the sequence of nucleotides 1787-2505, which was taken from the gene of Taq DNA polymerase, and which is identical to the nucleotide sequence 1781-2499 of Taq DNA polymerase gene.

Properties of a Chimeric Thermostable Enzyme of the Present Invention

A chimeric thermostable enzyme of the present invention represents a significant improvement over thermostable DNA polymerases described in the literature. In particular, the DNA polymerase of the invention provides the following combination of properties:
1. High efficiency of amplification of long DNA sequences. The efficiency of the chimeric enzyme is at least 5 times as high as that of Taq DNA polymerase and is no less than that of Tth DNA polymerase (Example 3).
2. High sensitivity to the presence of a mismatched nucleotide at the 3′ primer end. The chimeric enzyme is at least 6-fold more sensitive to the presence of a mismatch at the 3′-end of the primer than Tth DNA polymerase and is no less sensitive than Taq DNA polymerase (Example 4).
3. High specificity of DNA amplification in PCR. The chimeric enzyme shows much higher specificity in PCR-based amplification of DNA than Tth DNA polymerase and no less specificity than Taq DNA polymerase (Example 5); and
4. The DNA polymerase can be easily and efficiently expressed to a high level in a recombinant expression system, thereby facilitating commercial production of the enzyme (Example 2).

The combination of properties possessed by the chimeric thermostable enzyme of the invention is particularly useful in polymerase chain reactions, and provides significantly improved results. The present invention also encompasses a kit for use in PCR which may include the chimeric thermostable enzyme of the invention, a reaction buffer and dNTPs. Primers and double stranded template DNA specific to the reaction may also be included in the kit. Such a method of DNA amplification may include the following steps:
a) providing a reaction mixture comprising the chimeric thermostable enzyme of the invention, a reaction buffer, dNTPs, primers and double stranded template DNA;
b) heating the reaction mixture to separate the template DNA;
c) cooling the reaction mixture to allow bonding of the primers to the template DNA;
d) heating the reaction mixture to cause annealing of dNTPs catalysed by the chimeric thermostable enzyme;
e) repeating steps b) to d) to make multiple copies of template DNA.

The properties of the chimeric thermostable enzyme of the invention are illustrated below in the accompanying Examples.

### FIGURES

The figures referred to in the Examples are described more fully below.

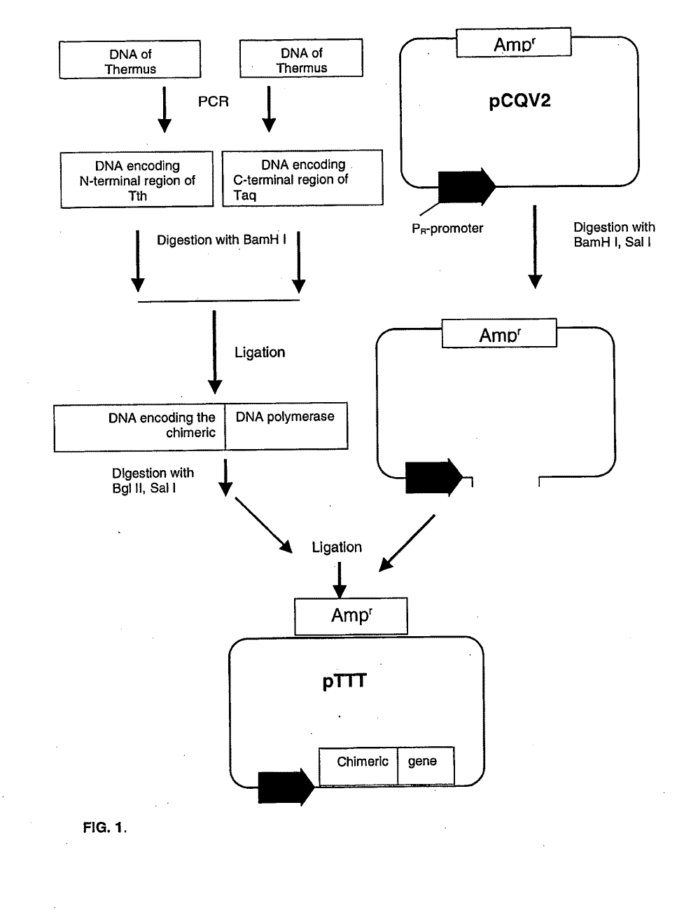

FIG. 1. Scheme illustrating steps in construction of plasmid pTTT, which contains the chimeric gene of the chimeric polymerase of the invention (described in detail in Example 1).

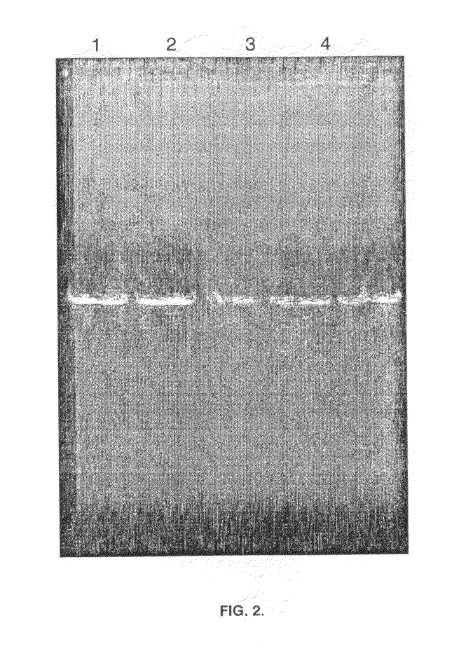

FIG. 2. Electrophoretic analysis of PCR products, which compares the yield of 2500-bp DNA fragment obtainable by PCR amplification with Taq DNA polymerase, Tth DNA polymerase and the chimeric thermostable enzyme of this invention and indicates that 0.5 U of the chimeric enzyme has the efficiency of PCR amplification similar to 0.5 U of Tth polymerase and 2.5 U of Taq polymerase. 2500-bp DNA fragment was amplified with 0.5 U of Tth (lane 1); 0.5 U of the chimeric enzyme (lane 2); 0.5 U, 1.5 U, 2.5 U of Taq polymerase (lanes 3, 4, 5 correspondingly) (described in detail in Example 3).

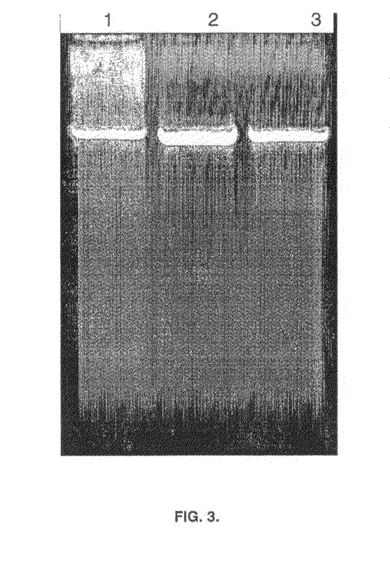

FIG. 3. Electrophoretic analysis of PCR products obtained in the presense of considerable quantity of E. coli DNA, which compares the specificity of PCR amplification reactions performed with Taq DNA polymerase, Tth DNA polymerase and the chimeric thermostable enzyme and indicates that chimeric enzyme shows much higher specificity in PCR than Tth and no less specificity than Taq polymerase. The reactions were performed with 3.5 U of Tth (lane 1), 3.5 U of the chimeric enzyme (lane 2) and 3.5 U of Taq DNA polymerase (lane 3) (described in detail in Example 5).

### EXAMPLES

The Examples relate to the production and testing of a chimeric thermostable enzyme of the invention. The Examples are illustrative of, but not binding on, the present invention. Any methods, preparations, solutions and such like, which are not specifically defined, may be found in Sambrook et al. All solutions are aqueous and made up in sterile, deionised water, unless otherwise specified. All enzymes were obtained from the Bioline Limited (London, GB)

### Example 1

### Construction of a Chimeric Gene and an Expression System

A chimeric gene was constructed, comprising a portion of the Tth DNA polymerase gene and a portion of the Taq DNA polymerase gene. In more detail, the procedure was as follows, in this Example.

A fragment of Tth DNA polymerase gene [U.S. Pat. No. 5,618,711], representing amino acids 4 to 597, was obtained by PCR amplification of total Thermus thermophilus DNA, primed by the two synthetic DNA primers PrTTH1 and PrTTH2 (below). Total DNA from Thermus thermophilus was isolated by the phenol deproteinisation method. The primers used were:

[SEQ ID NO 3]

PrTTH1 5′- ATAGATCTGATGCTTCCGCTCTTTGA -3′

[SEQ ID NO 4]

PrTTH2 5′- GGCCCGGCGGATCCTCTGGCCCAA -3′

Upstream primer PrTTH1 is homologous to wild type DNA starting at codon 4; this primer is designed to incorporate a Bgl II site into the amplified DNA product. Downstream primer PrTTH2 is homologous to codons 592-599 on the non-coding strand of the wild-type gene encoding Tth DNA polymerase and includes a BamH I site.

PCR was performed using a DNA Thermal Cycler 480 (Perkin-Elmer-Cetus). The reaction mixture (50 mkL) contained 67 mM Tris-HCl (pH 8.8), 16.6 mM (NH₄)₂ SO₄, 0.01% Tween-20, 0.2 mM of each dNTP's, 1.5 mM MgCl₂, 10 μmol of each primer, 100 ng of DNA as a template, and 5 U of Taq DNA polymerase. The reaction included 25 cycles: 94° C. for 30 s; 58° C. for 30 s; 72° C. for 100 s.

A DNA fragment of Taq DNA polymerase gene [Lawyer, F. C. et al., J. Biol. Chem., V.261, P. 6427-6437], encoding amino acids 592 to 832 was obtained by PCR amplification of total Thermus aquaticus YT1 DNA, primed by the two synthetic DNA primers PrTAQ1 and PrTAQ2 (below). Total DNA from Thermus aquaticus YT1 was isolated by the phenol deproteinisation method [Sambrook et al.]. The primers used were:

[SEQ ID NO 5]

PrTAQ1 5′- CAGAGGATCCGCCGGGCCTTCA -3′

[SEQ ID NO 6]

PrTAQ2 5′- AAGTCGACTCACTCCTTGGCGGAGAGCCA -3′

Upstream primer PrTAQ1 is homologous to wild type Thermus aquaticus YT1 DNA [Lawyer et al.] starting at codon 592 of the DNA polymerase gene and includes a BamH I site. Downstream primer PrTAQ2 is homologous to codons 827-832 on the other strand of the wild-type gene encoding Thermus aquaticus DNA polymerase and is designed to incorporate a SalG I site and a stop codon into the amplified fragment.

PCR was performed using a DNA Thermal Cycler 480 (Perkin-Elmer-Cetus). The reaction mixture (50 mkL) contained 67 mM Tris-HCl (pH 8.8), 16.6 mM (NH₄)₂SO₄, 0.01% v/v Tween-20, 0.2 mM of each dNTP's, 1.5 mM MgCl₂, 10 pmol of each primer, 100 ng of DNA as a template, and 5 U of Taq DNA polymerase. The reaction included 25 cycles: 94° C. for 30 s; 58° C. for 30 s; 72° C. for 150 s.

The amplified fragments (from Tth and Taq genes) were purified by 2% w/v agarose-gel electrophoresis, phenol extraction and were precipitated by ethanol. They were then digested with restriction endonuclease BamH I and ligated. The chimeric DNA fragment consisting of the Tth and Taq DNA fragments was obtained as a result of the manipulations.

The chimeric DNA fragment was purified by 1.5% w/v agarose-gel electrophoresis and phenol extraction, and was then precipitated by ethanol. The fragment was digested with restriction endonucleases Bgl II and SalG I and ligated into plasmid pCQV2 [Queen, C., J. Mol. Appl. Genet., V.2, P.1-10] which had been digested with the BamH I and SalG I restriction enzymes and previously treated with calf intestinal alkaline phosphatase [Sambrook et al.]. As a result, the chimeric gene encoding the chimeric DNA polymerase was cloned into pCQV2 under the control of the P_{R}-promoter.

Ligation was conducted with T4 DNA ligase in a 50 mkL volume containing 200 ng vector (plasmid pCQV2) and 200 ng of the insert. E. coli JM 109 cells were transformed with the ligation mixture according to the method of Dower et al. [Dower et al., Nucl. Acid. Res., V.16 (1988), P. 1127]. Transformed cells were grown on LB medium at 30° C. Clones were selected from ampicillin resistant colonies and checked to determine which ones contained the chimeric DNA polymerase gene insert.

Selected positives clones were assayed for production of protein of the corresponding MW by 12% SDS-polyacrylamide gel electrophoreses [Laemmli U., Nature V.227 (1970), P.680-685]. The cells were grown to an optical density of A₆₀₀=0.4 in 500 ml of LB medium containing ampicillin (75 mkg/ml) at 30° C. Heating to 42° C. induced expression of the cloned gene. The cells were further incubated for 4 h at 42° C. Cells were harvested by centrifugation and the enzyme was partially purified as follows.

All samples were isolated at 4° C. Cells (0.5 g) were suspended in 2 ml of buffer A (20 mM K-phosphate pH 7.0, 2 mM DTT, 0.5 mM EDTA) containing 0.2M NaCl and 0.1 mM phenylmethylsulphonyl-fluoride (PMSF). The cells were disrupted by ultrasonic disintegration (MSE, 150 wt) at maximum amplitude for 15 sec (3 impulses, each for 5 sec) with cooling on ice. The suspension was centrifuged at 20,000 g, the supernatant collected, and 5% v/v polyethylenimine was added to a final concentration of 0.1% v/v. The resulting precipitate was separated by centrifugation, and the supernatant removed. The supernatant proteins were then precipitated by solid ammonium sulfate at 75% saturation. The polymerase-containing precipitate was collected by centrifugation at 20,000 g, dissolved in 3 ml of buffer A, containing 0.1 M NaCl and 0.2% Tween-20, then heated for 5 minutes at 75° C. and centrifuged (10 min, 20,000 g). Denatured proteins were discarded and supernatant was assayed by its ability to perform PCR. A plasmid was isolated and purified from cells in which truncated chimeric polymerase was active in PCR.

PCR assays were conducted using a DNA thermal cycler 480 (Perkin Elmer-Cetus). The reaction mixture (50 mkL) contained 67 mM Tris-HCl (pH 8.8 at 25° C.), 16.6 mM (NH₄)₂SO₄, 0.01% Tween-20, 0.2 mM each dNTP, 1.5 mM MgCl₂, 10 pmol each primer (Pr.lambda.1: 5′-GATGAGTTCGTGTCCGTACAACTGG-3′[SEQ ID NO 7] and Pr.lambda.2: 5′-GGTTATCGAAATCAGCCACAGCGCC-3′[SEQ ID NO 8]), 50 ng template lambda DNA and 2.mkl of the above supernatant containing the enzyme. 30 cycles of the following cycle was carried out; 94° C. for 30 seconds, 57° C. for 40 seconds and 72° C. for 30 seconds.

Plasmid DNA was isolated from cells which produced a chimeric enzyme that was active in PCR. The plasmid was purified, and designated pTTT. The nucleotide sequence encoding the chimeric enzyme was verified by sequencing. The construction of pTTT is shown in FIG. 1.

### Example 2

### Preparation of Chimeric Thermostable Enzyme Using an Expression Vector (Plasmid pTTT)

E. coli JM 109 cells were transformed with the plasmid pTTT according to the method of Dower et al. [1988, Nucl. Acid. Res., V.16, P.6127]. The transformed cells were grown to an optical density of A.₆₀₀=0.4 in 7 L of LB medium containing ampicillin (75 mkg/ml) at 30° C. Expression of the chimeric gene encoding the chimeric polymerase was induced by heating to 42° C. The cells were further incubated for 7 h at 42° C. Cells were harvested by centrifugation.

The cells (35 g) were suspended in 70 ml of buffer A (20 mM K-phosphate pH 7.0, 2 mM DTT, 0.5 mM EDTA) containing 0.2M NaCl and 0.1 mM PMSF. The cellular walls were disrupted with an ultrasonic disintegrator (MSE, 150 wt) at maximum amplitude for 15 minutes (30 impulses, each for 30 sec) and with cooling on ice. The suspension was then centrifuged at 40,000 g, the pellet discarded, and 5% polyethylenimine was added to the supernatant to a final concentration of 0.1%. The precipitate was separated by centrifugation, and the remaining proteins precipitated with ammonium sulfate at 45% saturation. The resulting polymerase-containing precipitate was collected by centrifugation at 20,000 g and dissolved in buffer A (30 ml) containing 0.1 M NaCl and 0.2% Tween-20, heated for 15 minutes at 75° C. in the presence of 10 mM MgCl₂, and centrifuged for 10 minutes at 40,000 g.

The supernatant was loaded on to a (2.5×20 cm) phosphocellulose P-11 column (Whatman) equilibrated in buffer A containing 0.1 M NaCl, and washed out with the same buffer. The proteins were eluted with a linear gradient of NaCl concentrations ranging from 100 to 500 mM in buffer A. The gradient volume was 800 ml, and the flow rate was 60 ml/h. Polymerase was eluted at NaCl concentrations ranging from 280 to 330 mM.

The fractions were tested for polymerase activity, assayed via inclusion of the radioactive-labeled nucleotide ³²P(DATP) into the acid-insoluble pellet [Myers T. W., Gelfand D. H., (1991) Biochemistry, v30, N31, p7661-7666].

Specifically, the amount of the enzyme that incorporated 10 nmol of deoxynucleotide triphosphates into the acid-insoluble fraction within 30 minutes under conditions described below was taken as one unit of activity. The reaction mixture (50 mkL) contained 25 mM N-Tris [Hydroxymethyl]methyl-3-aminopropanesulphonic acid (TAPS), pH 9.3, 50 mM KCl, 2 mM MgCl₂; 1 mM β-mercaptoethanol; 0.2 mM of each dNTP's, 1 mkCi ³²P(dATI), and 12.5 mkg of activated salmon sperm DNA. The polymerase activity was determined at 73° C. (Salmon sperm DNA (12.5 mg/ml) was activated in 10 mM Tris-HCl (pH 7.2) containing 5 mM MgCl₂ with pancreatic DNase I (0.03 U/ml) at 4° C. for 1 h and then heated at 95° C. for 5 minutes.)

Fractions containing the polymerase activity were combined, dialyzed against buffer A containing 50 mM NaCl and loaded on to a column (0.6×6 cm) of DEAE-cellulose (Whatman) equilibrated with same buffer. The proteins were eluted with a linear gradient of NaCl concentrations ranging from 50 to 250 mM in buffer A. The gradient volume was 150 ml, and the flow rate was 15 ml/h. The polymerase was eluted at 150-200 mM NaCl. Polymerase activity was assayed as described above. Yield of polymerase activity was 1,475,000 units.

The purified enzymes were stored at −20° C. in the following buffer: 100 mM NaCl; 10 mM Tris HCl pH 7.5; 1 mM DTT; 0.2% Tween 20 and 50% (v/v) glycerol.

Homogeneity of the polymerase preparations was not less than 95% according to SDS electrophoresis data on a 10% polyacrylamide gel.

### Example 3

### Efficiency of PCR Amplification

The efficiency of PCR amplification by the Chimeric thermostable enzyme, Taq and Tth polymerases was estimated by amplification of 2500-bp DNA fragment.

PCR reactions were performed using a DNA thermal cycler 480 (Perkin Elmer-Cetus). The reaction mixture (50 mkL) contained 67 mM Tris-HCl (pH 8.8 at 25° C.), 16.6 mM (NH₄)₂SO₄, 0.01% Tween-20, 0.2 mM each dNTP, 1.5 mM MgCl₂, 10 pmol each primer (Pr.lambda.1: 5′-GATGAGTTCGTGTCCGTACAACTGG-3′[SEQ ID NO 7] and Pr.lambda.3: 5′-TGTTGACCTTGCCTGCAGCAACGC-3′[SEQ ID NO 9]), 5 ng template lambda DNA. The reactions were performed with 0.5 U of Tth polymerase; or 0.5 U of the Chimeric polymerase; or 0.5 U, 1.5 U, 2.5 U of Taq polymerase. 26 cycles of the following cycle were carried out: 94° C. for 30 seconds, 57° C. for 40 seconds and 72° C. for 100 seconds.

The results are shown in FIG. 2, and indicate that 0.5 U of the chimeric enzyme of the invention has the efficiency of PCR amplification similar to 0.5 U of Tth polymerase and 2.5 U of Taq polymerase. Thus, the chimeric enzyme has at least 5 times higher efficiency in PCR than Taq polymerase.

### Example 4

### Sensitivity to the Presence of a Mismatched Nucleotide at the 3′ Primer End

Enzyme sensitivity of the Chimeric thermostable enzyme, Taq and Tth DNA polymerases to the presence of a mismatch at the 3′-end of a primer was estimated by comparing the amounts of DNA synthesized in PCR with the primers either containing or not the 3′-mismatching nucleotide. PCR amplification of the 500-bp phage lambda DNA fragment was performed with the primer pairs: Pr.lambda.1 [SEQ ID NO 7]/Pr.lambda.2 [SEQ ID NO 8]; Pr.lambda.12 (5′-GATGAGTTCGTGTCCGTACAACTGC) [SEQ ID NO 10]/Pr.lambda.2 [SEQ ID NO 8]; Pr.lambda.13 (5′-GATGAGTTCGTGTCCGTACAACTGA) [SEQ ID NO 11]/Pr.lambda.2 [SEQ ID NO 8]; Pr.lambda.14 (5′-GATGAGTTCGTGTCCGTACAACTGT) [SEQ ID NO 12]/Pr.lambda.2 [SEQ ID NO 8]. The primers Pr.lambda.1 and Pr.lambda.2 were complementary to the corresponding fragment of phage lambda DNA; the primers Pr.lambda12, Pr.lambda13 and Pr.lambda14 were identical to Pr.lambda1, except the 3′-terminal nucleotide. The reaction mixture (50 μl) contained 67 mM Tris-HCl (pH 8.8), 16.6 mM (NH₄)₂SO₄, 0.01% Tween-20, 0.2 mM of each dNTPs, 1.5 mM MgCl₂, 17 pmol of each primer, 15 ng of phage lambda DNA as a template, and 1.5 U of the Chimeric, or Taq, or Tth DNA polymerase. The reaction proceeded in 25 cycles: 94° C. for 45 s; 59° C. for 30 s; 72° C. for 30 s.

To estimate the amount of the synthesized DNA, [alpha-³²P]dATP was added to the reaction mixture (2 μCi/50 μl reaction mixture), and radioactivity of the acid-insoluble fraction was then determined. For this purpose, the reaction was performed, and 20 μl of the resulting mixture was applied on a GF/B filter (Whatman). The filter was washed with 10% trichloroacetic acid and dried. The radioactivity was determined with a Beckman LS 9800 scintillation counter using Ready-Solv HP scintillation liquid (Beckman).

The results are shown in Table 1, and indicate that the presence of mismatching nucleotide at the 3′-end of the elongated DNA strand decreased to the same extent the PCR amplification efficiency by both Taq DNA polymerase and Chimeric thermostable enzyme. Thus, the chimeric enzyme is no less sensitive to the presence of a mismatch than Taq DNA polymerase and is at least 6-fold more sensitive than Tth DNA polymerase (Table 1).

### Example 5

### Specificity of DNA Amplification in PCR

Specificity of PCR DNA amplification is a ratio of target product of amplification to total synthesized DNA. Enzyme specificity of the Chimeric thermostable enzyme, Taq and Tth DNA polymerases was estimated by amplification of 2500-bp phage lambda DNA fragment in the presence of considerable quantity of E. coli DNA.

PCR reactions were performed using a DNA thermal cycler 480 (Perkin Elmer-Cetus). The reaction mixture (50 mkL) contained 67 mM Tris-HCl (pH 8.8 at 25° C.), 16.6 mM (NH₄)₂SO₄, 0.01% Tween-20, 0.2 mM each dNTP, 1.5 mM MgCl₂, 20 pmol each primer (Pr.lambda.1 [SEQ ID NO 7] and Pr.lambda.3 [SEQ ID NO 9]), 5 ng template lambda DNA, and 300 ng of E. coli DNA. The reactions were performed with 3.5 U of Chimeric thermostable enzyme, Tth or Taq DNA polymerase. 30 cycles of the following cycle was carried out: 94° C. for 30 seconds, 57° C. for 40 seconds and 72° C. for 100 seconds.

The results are shown in FIG. 3, and indicate that chimeric enzyme shows much higher specificity in PCR-based amplification of DNA than Tth DNA polymerase and no less specificity than Taq DNA polymerase.

TABLE 1

Radioactive label incorporation into the 500-bp DNA fragment synthesized with Taq, Tth, or the chimeric thermostable enzyme by PCR with primers containing or not containing 3′-mismatching nucleotides (described in detail in Example 4).

TABLE 1

| | | | Student's error |
|---|---|---|---|
| | | Radioactivity, | (P = 0.05), |
| DNA polymerase | Primers | 10⁵ cpm | 10³ cpm |
| Tth | Prλ1/Prλ2 | 3.30 | 3.2 |
| | Prλ12/Prλ2 | 3.25 | 2.5 |
| | Prλ13/Prλ2 | 3.24 | 2.4 |
| | Prλ14/Prλ2 | 3.27 | 2.8 |
| Chimeric | Prλ1/Prλ2 | 3.28 | 2.9 |
| | Prλ12/Prλ2 | 0.54 | 1.7 |
| | Prλ13/Prλ2 | 0.51 | 1.2 |
| | Prλ14/Prλ2 | 0.53 | 1.1 |
| Taq | Prλ1/Prλ2 | 3.01 | 2.6 |
| | Prλ12/Prλ2 | 0.51 | 1.3 |
| | Prλ13/Prλ2 | 0.48 | 1.2 |
| | Prλ14/Prλ2 | 0.49 | 2.0 |
| Background | | 0.009 | 0.2 |
| (without | | | |
| polymerase) | | | |

### REFERENCES

Patent Documents
U.S. Pat. No. 6,228,628; Mutant chimeric DNA polymerase, Gelfand D. H., Reichert F. L.
GB 2,344,591; Thermostable DNA polymerase, Kramarov V.M., Ignatov K.B., Hallinan J.P.
U.S. Pat. No. 4,965,188; Process for amplifying, detecting, and/or cloning nucleic acid sequences using a thermostable enzyme, Mullis K. B., Erlich H. A., Gelfand D. H., Horn G., Saiki R.K.
U.S. Pat. No. 5,618,711; Recombinant expression vectors and purification methods for Thermus thermophilus DNA polymerase, Gelfand D. H., Lawyer F. C., Stoffel S.
U.S. Pat. No. 5,079,352; Purified thermostable enzyme, Gelfand D. H., Stoffel S., Lawyer F. C., Saiki R. K.

Citations
Ohler L. D., and Rose E. A. (1992) Optimization of long-distance PCR using a transposon-based model system. PCR Methods Appl. 2: 51-59.
Ignatov K. B., Kramarov V. M., Chostyakova L. G. and Miroshnikov A. I. (1997) Factors determining different processivity of Tth and Taq DNA polymerases in amplification of phage X DNA. Mol. Biol. (Russ.) 31: 956-961.
Ignatov K. B., Kramarov V. M., Uznadze O. L. and Miroshnikov A. I. (1997) Tth DNA polymerase—mediated amplification of DNA fragments using primers with mismatches in the 3′-region. Bioorg. Khim. (Russ.) 23: 817-822.
Villbrandt B., Sobek H., Frey B. and Schomburg D. (2000) Domain exchange: chimeras of Thermus aquaticus DNA polymerase, Escherichia coli DNA polymerase I and Thermotoga neapolitana DNA polymerase. Protein Eng. 13: 645-654.
Barnes W. M. (1992) The fidelity of Taq polymerase catalyzing PCR is improved by an N-terminal deletion. Gene 112: 29-35.
Blanco L., Bernad A., Blasco M.A., and Salas M. (1991) A general structure for DNA-dependent DNA polymerases. Gene 100: 27-28.
Goodchild J., (1990) Conjugates of oligonucleotides and modified oligonucleotides: a review of their synthesis and properties. Bioconjugate Chemistry 1: 165-187.
Sambrook et al, (1989) “Molecular cloning—A Laboratory Manual”, Cold Spring Harbor Laboratory Press.
Lawyer F. C., Stoffel S., Saiki R. K., Myambo K., Drummond R., and Gelfand D. H. (1989) Isolation, characterization and expression in E. coli of the DNA polymerase gene from Thermus aquaticus. J. Biol. Chem. 264: 6427-6437.
Queen C. (1983) A vector that uses phage signals for efficient synthesis of proteins in E. coli. J. Mol. Appl. Genet. 2: 1-10.
Dower W. J., Miller J. F. and Ragsdale C. W. (1988) High efficiency transformation of E. coli by high voltage electroporation. Nucleic Acids Res. 16: 6127-6145.
Laemmli U.K. (1970) Cleavage of structural proteins assembly of the head of bacteriophage T4. Nature, 227: 680-685

1. A chimeric thermostable enzyme comprising an N-terminal region, a C-terminal region and a DNA polymerase domain, wherein the N-terminal region comprises an N-terminal region from a Thermus thermophilus (Tth) DNA polymerase I, the C-terminal region comprises a C-terminal region from a Thermus aquaticus (Taq) DNA polymerase I and the DNA polymerase domain comprises a portion of a DNA polymerase domain of the N-terminal region from Tth DNA polymerase I operably linked to a portion of a DNA polymerase domain of the C-terminal region from Taq DNA polymerase I.

2. A chimeric thermostable enzyme according to claim 1, wherein the N-terminal region from Tth DNA polymerase I further comprises a 5′ nuclease domain.

3. A chimeric thermostable enzyme according to claim 1, wherein the N-terminal region of the chimeric enzyme comprises an amino acid sequence from between positions 1 to 280 through to position n of Tth DNA polymerase I (SEQ ID No: 13), wherein n is between amino acids 555 to 601 of Tth DNA polymerase I and corresponds to an amino acid in position m of Taq DNA polymerase I (SEQ ID No: 14), wherein m is equal to n-2.

4. A chimeric thermostable enzyme according to claim 3, wherein the C-terminal region of the chimeric enzyme comprises an amino acid sequence from position m+1 through to 832 of Taq DNA polymerase I (SEQ ID No: 14).

5. A chimeric thermostable enzyme according to claim 1, wherein the amino acid sequence of the N-terminal region of the chimeric enzyme comprises an amino acid sequence from positions 4 to 600 of Tth DNA polymerase I (SEQ ID No: 13).

6. A chimeric thermostable enzyme according to claim 1 comprising an amino acid substitution of Asp for Glu at amino acid position 2 of the N-terminal region from Tth DNA polymerase I.

7. A chimeric thermostable enzyme according to claim 1 comprising an amino acid substitution of Leu for Ala at amino acid position 3 of the N-terminal region from Tth DNA polymerase I.

8. A chimeric thermostable enzyme according to claim 1 comprising the amino acid sequence of SEQ ID No: 1, or a fragment or variant thereof, wherein the fragment and variant exhibit DNA polymerase I activity.

9. A chimeric thermostable enzyme according to claim 8, wherein the variant has at least 92% sequence identity to SEQ ID No: 1.

10. A chimeric thermostable enzyme according to claim 8, wherein the fragment comprises residues 280 to 828 of SEQ ID NO: 1 or a variant thereof when aligned with SEQ ID NO: 1.

11. A nucleic acid encoding the chimeric thermostable enzyme according to claim 1.

12. A nucleic acid according to claim 11, comprising the nucleotide sequence of SEQ ID No: 2.

13. A recombinant DNA vector that comprises the nucleic acid of claim 11.

14. A host cell comprising the vector of claim 13.

15. A method of making a chimeric thermostable enzyme according to claim 1 comprising cultivating the host cell comprising a nucleic acid coding said enzyme under conditions for expression of the chimeric thermostable enzyme, and recovering said enzyme from the host cell.

16. A kit comprising the chimeric thermostable enzyme according to claim 1, reaction buffer and dNTPs.

17. A method of DNA amplification using the polymerase chain reaction comprising the steps of:
a) providing a reaction mixture comprising the kit of claim 16, primers and double stranded template DNA;
b) heating the reaction mixture to separate the template DNA;
c) cooling the reaction mixture to allow bonding of the primers to the template DNA;
d) heating the reaction mixture to cause annealing of dNTPs catalysed by the chimeric thermostable enzyme;
e) repeating steps b) to d) to make multiple copies of template DNA.


SEQUENCE LISTING (14 sequences)

SEQ ID NO: 1 - protein, 834 aa, Artificial sequence
  Chimeric DNA polymerase (Thermus thermophilus/ Thermus aquaticus)
MDLMLPLFEPKGRVLLVDGHHLAYRTFFALKGLTTSRGEPVQAVYGFAKSLLKALKEDGYKAVFVVFDAKAPSFRHEAYEAYKAGRAPTPEDFPRQLALIKELVDLLGFTRLEVPGYEADDVLATLAKKAEKEGYEVRILTADRDLYQLVSDRVAVLHPEGHLITPEWLWEKYGLRPEQWVDFRALVGDPSDNLPGVKGIGEKTALKLLKEWGSLENLLKNLDRVKPENVREKIKAHLEDLRLSLELSRVRTDLPLEVDLAQGREPDREGLRAFLERLEFGSLLHEFGLLEAPAPLEEAPWPPPEGAFVGFVLSRPEPMWAELKALAACRDGRVHRAADPLAGLKDLKEVRGLLAKDLAVLASREGLDLVPGDDPMLLAYLLDPSNTTPEGVARRYGGEWTEDAAHRALLSERLHRNLLKRLEGEEKLLWLYHEVEKPLSRVLAHMEATGVRRDVAYLQALSLELAEEIRRLEEEVFRLAGHPFNLNSRDQLERVLFDELRLPALGKTQKTGKRSTSAAVLEALREAHPIVEKILQHRELTKLKNTYVDPLPSLVHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 2 - DNA, 2505 bp, Artificial sequence
  Nucleotide sequence encoding the chimeric DNA polymerase (Thermus thermophilus/ Thermus aquaticus)
atggatctgatgcttccgctctttgaacccaaaggccgggtcctcctggtggacggccaccacctggcctaccgcaccttcttcgccctgaagggcctcaccacgagccggggcgaaccggtgcaggcggtctacggcttcgccaagagcctcctcaaggccctgaaggaggacgggtacaaggccgtcttcgtggtctttgacgccaaggccccctccttccgccacgaggcctacgaggcctacaaggcggggagggccccgacccccgaggacttcccccggcagctcgccctcatcaaggagctggtggacctcctggggtttacccgcctcgaggtccccggctacgaggcggacgacgttctcgccaccctggccaagaaggcggaaaaggaggggtacgaggtgcgcatcctcaccgccgaccgcgacctctaccaactcgtctccgaccgcgtcgccgtcctccaccccgagggccacctcatcaccccggagtggctttgggagaagtacggcctcaggccggagcagtgggtggacttccgcgccctcgtgggggacccctccgacaacctccccggggtcaagggcatcggggagaagaccgccctcaagctcctcaaggagtggggaagcctggaaaacctcctcaagaacctggaccgggtaaagccagaaaacgtccgggagaagatcaaggcccacctggaagacctcaggctctccttggagctctcccgggtgcgcaccgacctccccctggaggtggacctcgcccaggggcgggagcccgaccgggaggggcttagggccttcctggagaggctggagttcggcagcctcctccacgagttcggcctcctggaggcccccgcccccctggaggaggccccctggcccccgccggaaggggccttcgtgggcttcgtcctctcccgccccgagcccatgtgggcggagcttaaagccctggccgcctgcagggacggccgggtgcaccgggcagcagaccccttggcggggctaaaggacctcaaggaggtccggggcctcctcgccaaggacctcgccgtcttggcctcgagggaggggctagacctcgtgcccggggacgaccccatgctcctcgcctacctcctggacccctccaacaccacccccgagggggtggcgcggcgctacgggggggagtggacggaggacgccgcccaccgggccctcctctcggagaggctccatcggaacctccttaagcgcctcgagggggaggagaagctcctttggctctaccacgaggtggaaaagcccctctcccgggtcctggcccacatggaggccaccggggtacggcgggacgtggcctaccttcaggccctttccctggagcttgcggaggagatccgccgcctcgaggaggaggtcttccgcttggcgggccaccccttcaacctcaactcccgggaccagctggaaagggtgctctttgacgagcttaggcttcccgccttggggaagacgcaaaagacaggcaagcgctccaccagcgccgcggtgctggaggccctacgggaggcccaccccatcgtggagaagatcctccagcaccgggagctcaccaagctcaagaacacctacgtggaccccctcccaagcctcgtccacccgaggacgggccgcctccacacccgcttcaaccagacggccacggccacggggaggcttagtagctccgaccccaacctgcagaacatccccgtccgcacccccttgggccagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgccgggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtga

SEQ ID NO: 3 - DNA, 26 bp, Artificial sequence
  Primer
atagatctgatgcttccgctctttga

SEQ ID NO: 4 - DNA, 24 bp, Artificial sequence
  Primer
ggcccggcggatcctctggcccaa

SEQ ID NO: 5 - DNA, 22 bp, Artificial sequence
  Primer
cagaggatccgccgggccttca

SEQ ID NO: 6 - DNA, 29 bp, Artificial sequence
  Primer
aagtcgactcactccttggcggagagcca

SEQ ID NO: 7 - DNA, 25 bp, Artificial sequence
  Primer
gatgagttcgtgtccgtacaactgg

SEQ ID NO: 8 - DNA, 25 bp, Artificial sequence
  Primer
ggttatcgaaatcagccacagcgcc

SEQ ID NO: 9 - DNA, 24 bp, Artificial sequence
  Primer
tgttgaccttgcctgcagcaacgc

SEQ ID NO: 10 - DNA, 25 bp, Artificial sequence
  Primer
gatgagttcgtgtccgtacaactgc

SEQ ID NO: 11 - DNA, 25 bp, Artificial sequence
  Primer
gatgagttcgtgtccgtacaactga

SEQ ID NO: 12 - DNA, 25 bp, Artificial sequence
  Primer
gatgagttcgtgtccgtacaactgt

SEQ ID NO: 13 - protein, 834 aa, Thermus thermophilus
  DNA Polymerase
MEAMLPLFEPKGRVLLVDGHHLAYRTFFALKGLTTSRGEPVQAVYGFAKSLLKALKEDGYKAVFVVFDAKAPSFRHEAYEAYKAGRAPTPEDFPRQLALIKELVDLLGFTRLEVPGYEADDVLATLAKKAEKEGYEVRILTADRDLYQLVSDRVAVLHPEGHLITPEWLWEKYGLRPEQWVDFRALVGDPSDNLPGVKGIGEKTALKLLKEWGSLENLLKNLDRVKPENVREKIKAHLEDLRLSLELSRVRTDLPLEVDLAQGREPDREGLRAFLERLEFGSLLHEFGLLEAPAPLEEAPWPPPEGAFVGFVLSRPEPMWAELKALAACRDGRVHRAADPLAGLKDLKEVRGLLAKDLAVLASREGLDLVPGDDPMLLAYLLDPSNTTPEGVARRYGGEWTEDAAHRALLSERLHRNLLKRLEGEEKLLWLYHEVEKPLSRVLAHMEATGVRRDVAYLQALSLELAEEIRRLEEEVFRLAGHPFNLNSRDQLERVLFDELRLPALGKTQKTGKRSTSAAVLEALREAHPIVEKILQHRELTKLKNTYVDPLPSLVHPRTGRLHTRFNQTATATGRLSSSDPNLQNIPVRTPLGQRIRRAFVAEAGWALVALDYSQIELRVLAHLSGDENLIRVFQEGKDIHTQTASWMFGVPPEAVDPLMRRAAKTVNFGVLYGMSAHRLSQELAIPYEEAVAFIERYFQSFPKVRAWIEKTLEEGRKRGYVETLFGRRRYVPDLNARVKSVREAAERMAFNMPVQGTAADLMKLAMVKLFPRLREMGARMLLQVHDELLLEAPQARAEEVAALAKEAMEKAYPLAVPLEVEVGMGEDWLSAKG

SEQ ID NO: 14 - protein, 832 aa, Thermus aquaticus [sequence omitted]